(19) United States
(12) Patent Application Publication
(10) Pub. No.: US 2013/0254136 A1
(43) Pub. Date: Sep. 26, 2013

(54) CUSTOMIZABLE DISPENSING SYSTEMS AND DISPENSING SYSTEMS DELIVERING A DOSE OF FRAGRANCE UPON ACTUATION

(71) Applicant: GOJO INDUSTRIES, INC., Akron, OH (US)

(72) Inventors: Nick E Ciavarella, Seven Hills, OH (US); Matthew J. Archer, Aurora, OH (US); Todd Cartner, Uniontown, OH (US); Diane Hillman, North Canton, OH (US); Amanda Jo Copeland, Seville, OH (US)

(73) Assignee: GOJO Industries, Inc., Akron, OH (US)

(21) Appl. No.: 13/826,098

(22) Filed: Mar. 14, 2013

Related U.S. Application Data
(60) Provisional application No. 61/614,108, filed on Mar. 22, 2012.

(51) Int. Cl. A61L 9/14

(52) U.S. Cl. CPC A61L 9/14
USPC 705/500; 222/190; 222/209

(57) ABSTRACT

A customizable fluid product dispensing system includes a container including a volume of fluid product, a pump secured to the container and in fluid communication with the fluid product, and a cover assembly positioned over a portion of the pump and secured to the pump or container. The pump may include a plastic component infused with a fragrance for imparting the fragrance to the fluid dispensed from the system. The cover assembly may also include a plastic component with an infused fragrance for providing an air freshening function to the dispensing system. A vignette may be provided on the cover assembly, and may correspond to the fragrance of one or both of the pump and cover assembly. An air freshener base unit is provided to interact with the dispensing systems or with common desktop type dispensers.

### CROSS REFERENCE TO RELATED FILINGS

The present invention claims the benefit of the filing of U.S. Provisional Patent Application No. 61/614,108, filed Mar. 22, 2012, incorporated herein by reference.

### FIELD OF THE INVENTION

The system and methods of this disclosure relate generally to a customizable fluid product dispensing system. More particularly, the system and methods of this disclosure relate to a customizable fluid product dispensing system that dispenses a fluid product having a selected fragrance. In other embodiment, the invention relates to

### BACKGROUND OF THE INVENTION

Personal dispensers of various liquid products for use in the home or office are commercially available in a variety of forms. Hand sanitizers, hand cleaners, and hand lotions are often dispensed with dispensers having positive displacement pumps. These dispensers may include a collapsible cavity that, when collapsed, forces a liquid product from the container. Alternatively, the dispensers may include a compressible dome pump. These dispensers may advantageously dispense a unit dose of liquid product upon activation of their dispensing mechanisms to provide a consistent volume of product to a user. Regardless of the type of pump provided, the dispensers each include a product reservoir containing a volume of the fluid product that is in fluid communication with the pump. These dispensing systems are typically made to be disposable, and are intended to be discarded upon emptying of the product reservoir.

Dispensers for hand sanitizers, hand cleaners, and hand lotions are generally manufactured and sold in generic bottles or containers without extensive design or aesthetic features. Providing dispensers with a variety of aesthetic features to consumers creates of number of manufacturing and inventory issues that makes the practice unattractive to the manufacturers of the devices. For example, where a product has a limited shelf-life, manufacturing of a plurality of different designs often leads to increased waste due to expiration of unused product. However, the ability to customize or personalize consumer products has become increasingly popular and desirable in recent years. Consumers have shown an interest in the ability to create products that reflect their personal interests and tastes.

Thus, there is a need for a fluid product dispensing system that alleviates one or more of the deficiencies of the prior art discussed above.

### SUMMARY OF THE INVENTION

A first embodiment of this invention provides a personalized fluid product dispensing system comprising a container including a volume of a fluid product; and a pump adapted to be secured to the container, the pump including at least one plastic component that carries a substance for imparting a property to a dispensed fluid product.

A second embodiment provides a dispensing system as in the first embodiment, wherein said container includes a product reservoir and said at least one plastic component is a dip tube carrying a fragrance and extending downwardly from said pump into said product reservoir, such that said property imparted to said fluid product is a fragrance.

A third embodiment provides a dispensing system as in either the first or second embodiments, further comprising a cover assembly, wherein said container further comprises a neck and said cover assembly includes a base portion positioned over said neck and a top portion positioned over a discharge spout of said pump.

A fourth embodiment provides a dispensing system as in any of the first through third embodiments, wherein said top portion is movable relative to said base portion to allow actuation of said pump.

A fifth embodiment provides a dispensing system as in any of the first through fourth embodiments, wherein said cover assembly includes at least one plastic component having an infused fragrance, the cover assembly acting as an air freshener.

A sixth embodiment provides a dispensing system as in any of the first through fifth embodiments, wherein said property imparted to said fluid product is a fragrance, and said cover assembly includes a vignette that corresponds to the fragrance.

A seventh embodiment provides a dispensing system as in any of the first through sixth embodiments, wherein said container contains a volume of hand cleaner, hand sanitizer, or hand lotion.

An eighth embodiment provides a dispensing system as in any of the first through seventh embodiments, wherein said property imparted by said plastic component of said pump is selected from one or more of a fragrance, a color, a moisturizer, a sunscreen and an insect repellant.

A ninth embodiment of this invention provides a method of merchandising a fluid product dispensing system comprising the steps of offering a container for sale, the container including a volume of fluid to be dispensed and a cap; offering a plurality of pumps for sale, each of the pumps including a plastic component infused with a different fragrance adapted to impart the fragrance to the fluid product being dispensed; and offering a plurality of cover assemblies for sale, the plurality of cover assemblies having various colors to allow for personalization of the dispensing system, each cover assembly adapted to be secured to the pump or container.

A tenth embodiment provides a method as in the ninth embodiment, wherein the container includes a volume of hand sanitizer, hand cleaner, or hand lotion.

An eleventh embodiment provides a method as in either the ninth or tenth embodiments, wherein said plurality of cover assemblies each include a plastic component with an infused fragrance for providing an air freshening function, each of said plurality of cover assemblies providing a different fragrance.

A twelfth embodiment provides a method as in any of the ninth through eleventh embodiments, further comprising the step of offering a plurality of vignettes for sale, the vignettes including a plurality of images or graphics for personalization of the cover assembly, wherein the vignettes correspond to the fragrances of the pump.

A thirteenth embodiment provides a method as in any of the ninth through twelfth embodiments, wherein said plurality of pumps each include a dip stick extending downwardly from the pump and adapted to be in fluid communication with the fluid in the container, the dip stick having an infused fragrance to impart the fragrance to fluid passing therethrough.

A fourteenth embodiment provides a method as in any of the ninth through thirteenth embodiments, further comprising the step of offering a plurality of vignettes for sale, the vignettes including a plurality of images or graphics for personalization of the cover assembly.

A fifteenth embodiment of this invention provides a dispensing system providing a dose of fragrance upon actuation of a dispenser thereof that is actuated to dispense product, the dispensing system comprising an fragrance-delivering assembly defining a collapsible volume that decreases upon actuation of the dispenser portion, said fragrance-delivering assembly carrying a fragrance; and an outlet communicating with said collapsible volume such that the decreasing of the collapsible volume forces a does of air out at said outlet, said dose of air being scented by said fragrance.

A sixteenth embodiment provides a dispensing system as in the fifteenth embodiment, wherein the dispenser includes a container having a neck, and a pump having a discharge spout that is moved to actuate the dispenser, wherein said fragrance-delivering assembly includes a cover assembly including a base portion positioned over said neck and a top portion positioned over said discharge spout, said top portion and said base portion defining said collapsible volume, and said top portion being pressed toward said base portion to move said discharge spout and actuate said dispenser.

A seventeenth embodiment provides a dispensing system as in either the fifteenth or sixteenth embodiments, wherein the dispenser includes a container and the dispensing system further comprises a base unit defining said collapsible volume, said base unit including a bottle support mounted for reciprocal movement in said base unit, said container being supported on said bottle support such that actuation of the dispenser causes movement of said bottle support, said movement causing a decrease in said collapsible volume.

An eighteenth embodiment provides a dispensing system as in any of the fifteenth through seventeenth embodiments, wherein the fragrance is provided by an air freshener carried in the fragrance-delivering assembly.

A nineteenth embodiment provides a dispensing system as in any of the fifteenth through eighteenth embodiments, wherein the fragrance is carried by a plastic component of said fragrance-delivering assembly, said plastic component being infused with a fragrance.

A twentieth embodiment provides a dispensing system as in any of the fifteenth through nineteenth embodiments, wherein said outlet is an adjustable vent.

### BRIEF DESCRIPTION OF THE DRAWINGS

For a full understanding of the invention reference should be made to the following detailed description and the accompanying drawings, wherein:

FIG. 1 is a perspective view of a customizable dispensing system according to the concepts of the present disclosure and shown with a portion of the wall of the container removed to show the contents thereof.

FIG. 2 is a front elevational view of the system of FIG. 1 showing the pump and dip tube and showing a partial cross section at the interaction of a collar 24 and neck 22.

FIG. 3 is a side elevation view of the system of FIG. 1.

FIG. 4 is a perspective view of a dispenser including a container and a pump for dispensing fluid product, the dispenser being employed in the system of this invention.

FIG. 5 is a front elevation view of the embodiment of FIGS. 1-4, shown with a scent-specific vignette on the exterior and shown with a portion of the walls of a cover assembly 16 removed to show the interior thereof.

FIG. 6 perspective view of a second embodiment of a dispensing system of the present disclosure and shown with a portion of the walls of a cover assembly 116 removed to show the interior thereof.

FIG. 7 is a perspective view of an embodiment of a fragrance-delivering base for a dispenser such as that of FIG. 4 or a dispensing system such as that of FIGS. 1-3, 5 and 6.

FIG. 8 is a cross-section of the fragrance-delivering base, taken along the line 8-8 of FIG. 7 and showing an interaction of a rib and channel in an enlarged view therein.

FIG. 9 is a cross-section of the fragrance-delivering base, taken along the line 9-9 of FIG. 7.

### DETAILED DESCRIPTION OF THE ILLUSTRATIVE EMBODIMENTS

In one or more embodiments, the dispensing system of this disclosure may dispense hand sanitizer, hand cleaner, or hand lotion. In certain embodiments, the dispensing system may include a container containing a fluid product to be dispensed and a pump secured to and in fluid communication with the container. In one or more embodiments, the pump may include plastic components that are infused with a fragrance to impart a fragrance to the fluid product dispensed from the pump. In a particular embodiment, the pump includes a dip stick, or dip tube, extending into the fluid product contained within the container, and the dip stick is infused with the fragrance. In one or more embodiments, the dispensing system also includes a cover assembly positioned around a neck of the container. The cover assembly may also include plastic components infused with a fragrance so that the dispensing system also acts as an air freshener. In one or more embodiments, a vignette may be applied to a portion of the cover assembly, and may optionally provide a pictorial representation of the fragrance of the pump, or the fragrance of the cover assembly.

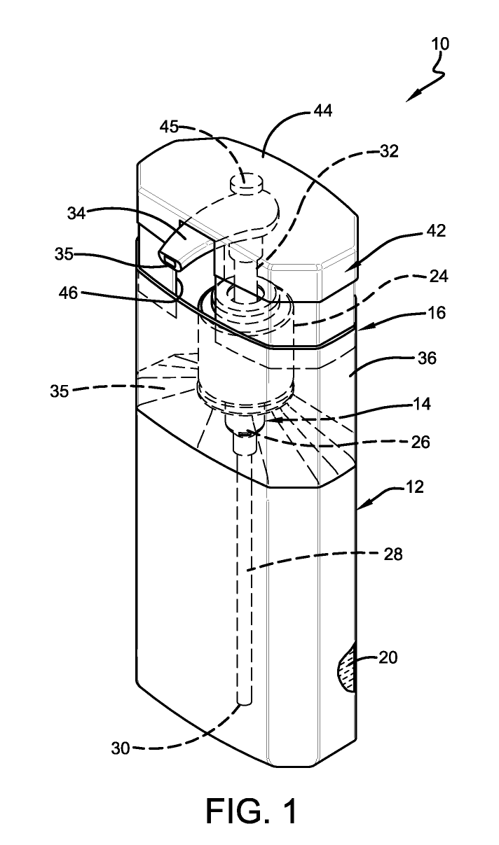

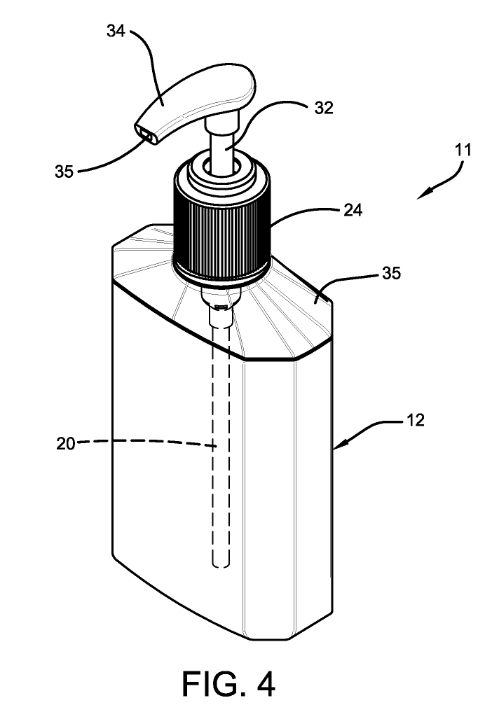

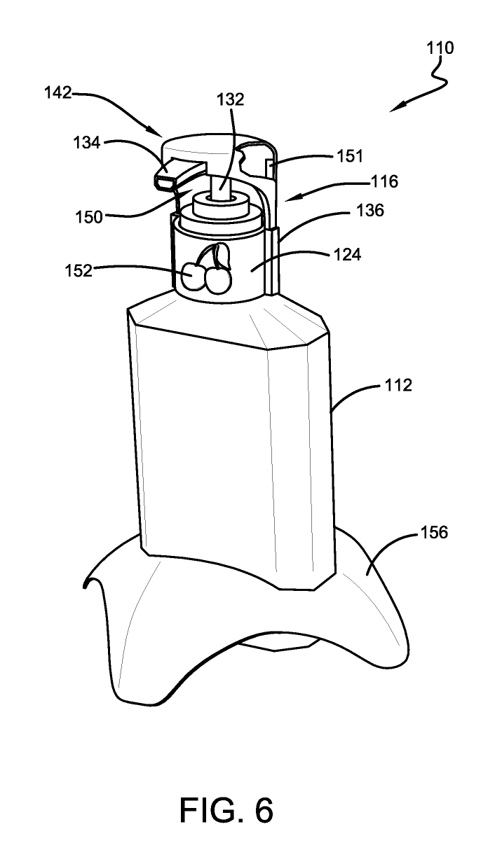

Referring now to FIG. 1, a dispensing system is shown, and is generally indicated by the numeral 10. Dispensing system 10 includes a product dispenser 11 (see FIG. 4) including a container 12 having a fluid product therein and a pump 14 in fluid communication with the container 12. The dispensing system further includes a cover assembly 16 positioned around the portions of the dispenser 11 that extend above the main body of the container 12. A base member (FIG. 6) may optionally be provided around a bottom portion of the container 12 to provide added support and stability to the dispensing system 10. It will be appreciated by those skilled in the art that a variety of types and configurations of base members may be utilized with the dispensing system 10.

The container 12 includes a product reservoir 20 containing the fluid product to be dispensed, and a neck 22 extending upwardly from the product reservoir. The cover assembly 16 is positioned around the neck 22. In one or more embodiments, the product reservoir 20 contains a hand sanitizer, a hand cleaner, or a hand lotion therein. In certain embodiments, the product reservoir 20 may be made of a transparent material so that the fluid product, and the amount of fluid product remaining in the product reservoir 20, is visible to a consumer. The product reservoir 20 may be provided in any desired size and shape to meet consumer demands and to satisfy manufacturing considerations. In one or more embodiments, the neck 22 may be formed integrally with the product reservoir 20 and may include external threads adapted to mate with the threads of a cap or pump, as will be described in greater detail below.

In one or more embodiments, the pump 14 is a positive displacement pump, as is known to those skilled in the art. In certain embodiments, the pump 14 includes a collar 24 having internal threads adapted to mate with threads provided by the neck 22. A collapsible cavity 26 of the pump 14 is carried by the collar 24 and adapted to extend through the neck 22 and into the container 12. A dip tube 28 extends downwardly from the collapsible cavity 26 with a distal end 30 of the dip tube 28 positioned proximate a bottom of the container 12. Though the present embodiment shown and described includes a threaded engagement between the neck 22 and the collar 24, it will be appreciate that this invention is not limited to such and engagement, and any structure for selectively engaging the pump 14 and the container 12 may be practiced.

As will appreciated by those skilled in the art, the dip tube 28 is in fluid communication with the container 12 and the collapsible cavity 26 to allow a volume of fluid to be drawn into the collapsible cavity following actuation of the pump. A piston 32 extends upwardly from the collapsible cavity 26 and a discharge spout 34 may extend from the top of the piston 32. The piston 32 and discharge spout 34 are in fluid communication with the collapsible cavity 26 to allow a volume of fluid to be dispensed when the piston and discharge spout are pressed downwardly to decrease the volume of the collapsible cavity. Valves may be provided within the pump to regulate flow to ensure that a dose of product is expelled from and then drawn into the collapsible cavity of the pump upon reciprocal movement of the discharge spout. A biasing mechanism, such as, for example, a spring, may be provided in pump 14 to bias the piston and discharge spout in an unactuated and upward position. Thus, reciprocal movement of the discharge spout 34 results in the dispensing of product from pump 14 and draws a dose of fluid into pump 14 for future dispensing. This type of pump is well known, and various design variations may be employed. For example, in certain embodiments, the pump 14 may be a foam pump, and therefore may be adapted to introduce air or other gas into a mixing chamber to generate a foam product, again, as known.

In one or more embodiments, the dip tube 28 may be made of a plastic material infused with a fragrance. The specific fragrance infused in the plastic dip tube 28 may be any suitable fragrance that consumers may find appealing, such as, for example, citrus, pine, or lavender scents. The fragrance infused dip tube 28 may transfer the selected fragrance to the fluid product (e.g. hand sanitizer) being dispensed. While the specific embodiment disclosed contemplates infusing the dip tube 28 with a fragrance, it is also contemplated that other plastic components of the pump 14 that contact the fluid being dispensed may be infused with a fragrance, either in addition to the dip tube or in lieu of the dip tube.

The specific fragrances infused in the plastic pump components may be any suitable fragrances that consumers may find appealing. Reference is made to U.S. Patent Publication 2004/0028779, which discloses a method of infusing plastic with a fragrance, and is incorporated herein by reference for that purpose. Reference is also made to U.S. Pat. No. 7,005,152, wherein a plastic component of a bottle includes fragranced oils to emit a perceivable fragrance. Notably, in the aforementioned publication and patent, the scented plastic serves merely to directly cause the user to perceive a scent directly from the scented plastic. In distinction, the present invention, the scented plastic components of the pump are infused with a fragrance in order to transfer the selected fragrance to the fluid product (e.g. hand sanitizer) being dispensed, such that the fragrance is then physically transferred to the user upon use of the fluid product.

It is also contemplated that the dip tube or other plastic components of the pump that come into contact with the liquid being dispensed may be made of a plastic material that carries other materials and/or properties, as may be apparent to those skilled in the art. The plastic components may be infused with or coated with a substance that is adapted to impart the desired properties or additives to the fluid product being dispensed. For example, in certain embodiments, the dip tube or other pump components may be coated or infused with a substance that imparts a color to the liquid being dispensed. In other embodiments, the dip tube or other plastic pump components may be coated or infused with a substance that imparts a moisturizer to the liquid being dispensed. Similarly, the dip tube or other plastic pump components may be coated or infused with a substance that imparts sunscreen or insect repellants to the liquid being dispensed.

The cover assembly 16 is adapted to be received over the exposed portion of the pump 14 above container 12. In particular embodiments the cover assembly 16 is received over the neck 22 of the container 12 and the collar 24 around the neck 22. The cover assembly 16 may be secured to the pump or container 12 by any method or mechanism known to those skilled in the art. In particular embodiments, the cover assembly 16 is supported at a bottom edge by the shoulder 35 of the container 12.

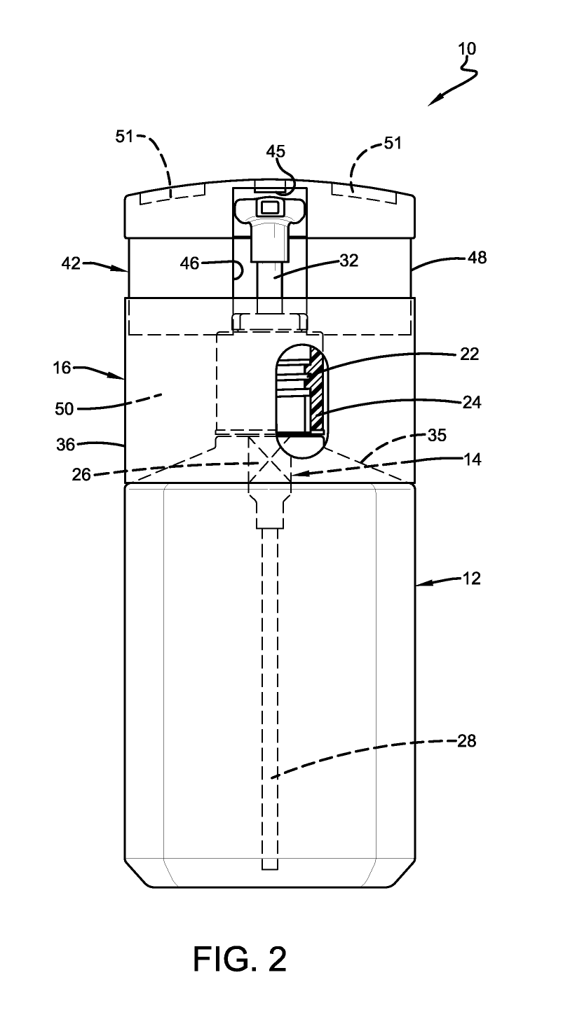

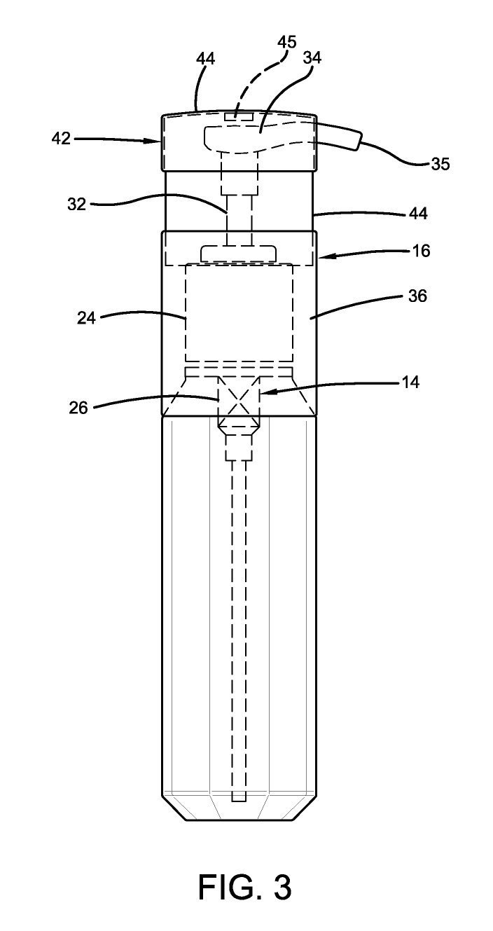

In certain embodiments, the cover assembly 16 includes a base portion 36 that is adapted to fit around the collar 24 (and thus also the neck 22) and rest on the shoulder 35 of the container 12. The base portion 36 may be shaped to provide an aesthetically pleasing dispensing system 10, and may therefore include any number of non-functional features. In certain embodiments, the base portion 36 may be designed to appear as an extension of the container 12, giving the impression of continuity between the two components, thus providing a very streamlined and attractive appearance, as shown in FIGS. 1-5. In other embodiments, a base portion 136 fits more intimately about a collar 124 (and neck, not shown/numbered but substantially as shown in FIG. 2), as shown in the dispensing system 110 of FIG. 6, wherein like parts receive like numerals to those of FIGS. 1-5, though increased by one hundred, thus having a container 112 and pump (not shown) held by a collar 124, with a piston 132 and discharge spout 134 extending therefrom.

Referring back to the embodiment of FIGS. 1-5, the cover assembly 16 further includes a top portion 42 adapted to be received over a portion of the discharge spout 34. The top portion 42 includes a top wall 44 that is positioned over the discharge spout 34, and further includes a slot 46 through which the discharge spout 34 extends to provide its dispensing outlet outside of the footprint of the cover assembly to permit a user's hand to be positioned there under. In some embodiments, a pad 45 is provided on the inside surface of the top wall 44 to cushion the contact between the top wall 44 and the spout. The slot 46 allows the discharge spout 34, particularly the dispensing outlet 35 thereof, to extend beyond the foot print of the top portion 42 to permit a user's hand to access an area below the dispensing outlet 35. Downwardly extending sidewalls 48 extending from the top wall 44 are adapted to be telescopically received by the base portion 36. The top portion 42 and base portion 36 are movable relative to one another. As will appreciated as those skilled in the art, the slidable engagement between the top portion 42 and the base portion 36 allows the top portion 42 to compress the discharge spout 34 and piston 32 of the pump 14, thereby causing actuation of the pump and dispensing of the fluid product. In one or more embodiments, the one of the base portion 36 and top portion 42 may include a slot or channel, while the other of the base portion or top portion includes a flange or rib adapted to be movably received in the slot or channel to provide the desired slidable engagement.

While a particular cover assembly 16 has been described and shown in FIGS. 1-5, it will be appreciated by those skilled in the art that many variations of this cover assembly are possible, so long as the cover assembly allows for movement of the discharge spout 34 and piston 32 upon actuation of the dispensing system. For example, FIG. 6 shows an alternative cover assembly 116 in which the base portion 136 fits more intimately around the collar 124, and a top portion 142 is vertically and reciprocally movable relative to the base.

In one or more embodiments, the base portion and top portion may be provided in aesthetically appealing colors to allow for customization of the dispensing system. In the same or other embodiments, the base portion and top portion of the cover assembly may be formed from fragrance infused plastic, similar to the dip tube discussed above. In certain embodiments, the fragrance of the cover assembly may be the same as, or may be complimentary to, the fragrance of the dip tube.

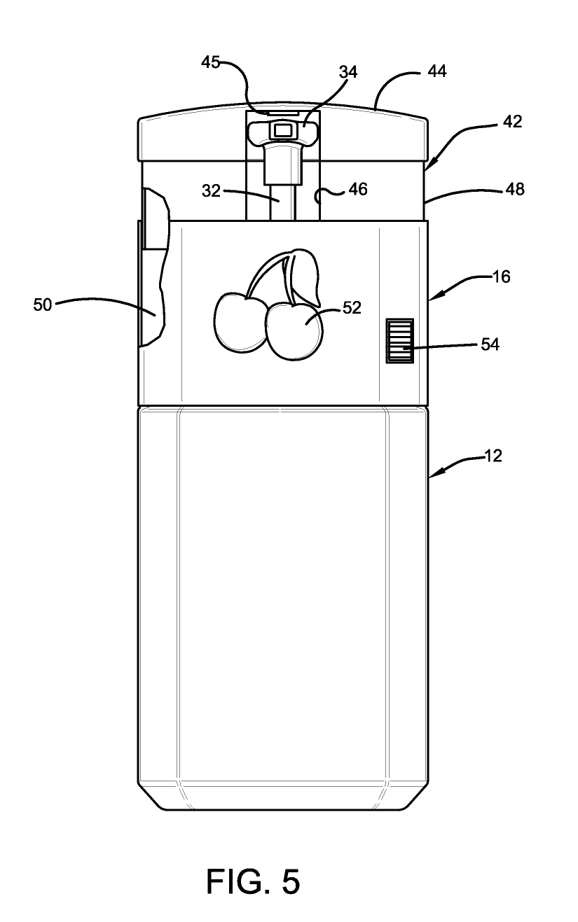

In one or more embodiments, the cover assembly may be provided with a vignette, as shown in FIGS. 5 and 6 at numerals 52 and 152. In certain embodiments, the vignette may be associated with the fragrance of one or both of the dip tube and cover assembly so as to provide an indicator of the included fragrance. The fragrance infused plastic of the cover assembly may act as an air freshener to provide a dispensing system 10 that is multifunctional.

Notably, in the specific structures shown, each cover assembly 16 and 116 defines a collapsible volume 50, 150 that decreases each time the top portion 42, 142 is pressed upon to dispense product. This forces a dose of air out from the collapsible cover assembly 16, 116, and, in embodiments where portions of the cover assembly are infused with fragrance, a “puff” (or dose) of fragrance fills the immediate area around the dispenser system 10, 110. The general concavity defining the collapsible volume 150 shrinks and air is forced in the general direction that the concavity faces. In embodiments such as that in FIGS. 1-3, the slot 46 for receipt of the discharge spout 34 provides communication between the collapsible volume 50 and the general surroundings such that pressing on the top wall 44 to effect movement of the discharge spout 34 forces a dose of fragrance out of the collapsible volume. In other embodiments, as schematically represented in FIG. 5, the structures defining the cover assembly 16 include one or more vents 54 so that the dose of fragrance is directed through the vent. Such vents can be adjustable as to flow rate and direction of the dose of fragrance. In other embodiments, an air freshener 51 (FIG. 2) or 151 (FIG. 6) could be employed in the collapsible volume 50, 150 to deliver the fragrance. This could be in addition to or in lieu of a scented plastic component.

In one or more embodiments, a consumer may purchase a container from a point of purchase with a disposable cap or removable tamper-evident seal (not shown) positioned over the open top of the container. The consumer may then select and purchase a pump having a desired fragrance (or no fragrance) from a plurality of pumps and fragrances made available. In one or more embodiments, the consumer may then select and purchase a cover assembly from a plurality of cover assemblies having a variety of colors and/or fragrances (or no fragrance). The container may, in certain embodiments, include a base member for added stability, and the base member could be selected by the consumer as well. This concept is shown in the embodiment of FIG. 6, at base member 156.

In certain embodiments, the cover assembly may include a vignette associated with the fragrance of either the cover assembly or the pump. In other embodiments, a vignette may be selected and purchased separately from a plurality of vignettes made available. In some embodiments, the vignettes will be associated with the various fragrances of the available pumps and/or cover assemblies. For example, a dispensing system including a pump with an apple fragrance may include an apple vignette on the cover assembly.

The consumer may then remove the cap from the container, secure the pump to the container, and assemble the cover assembly on the pump and container to create a personalized dispensing system. Notably, the customizable dispensing system utilizes a universal container, thereby avoiding inventory and manufacturing issues where the container includes a product having a limited shelf life.

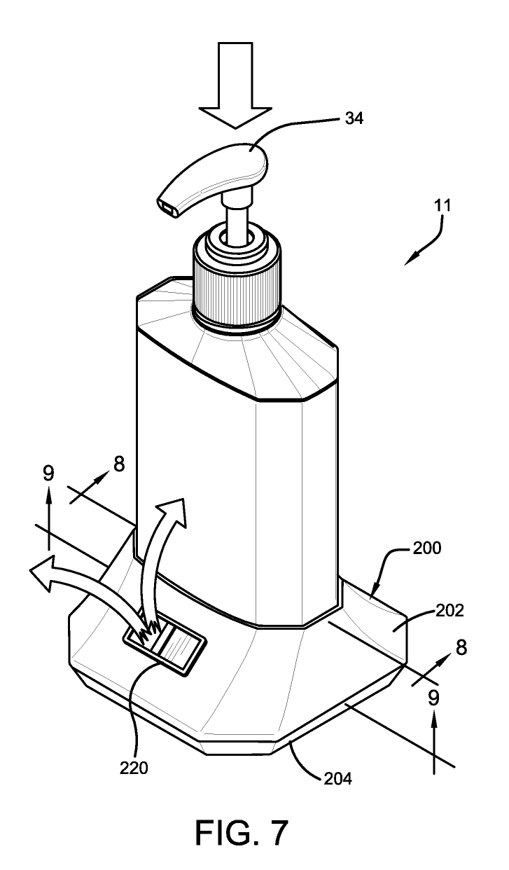

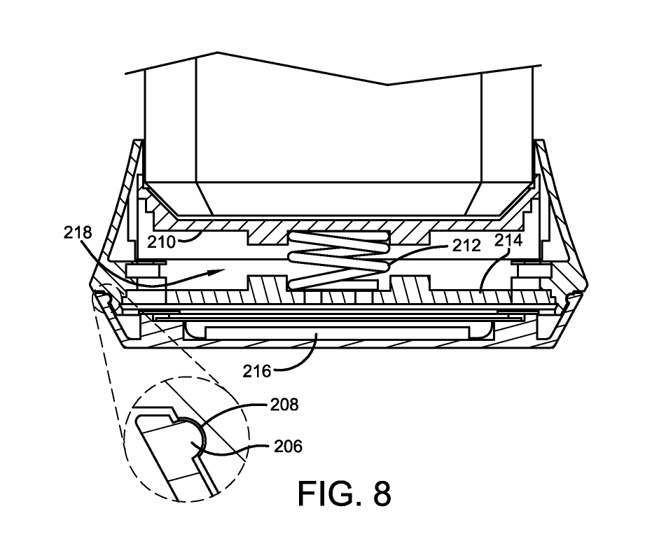

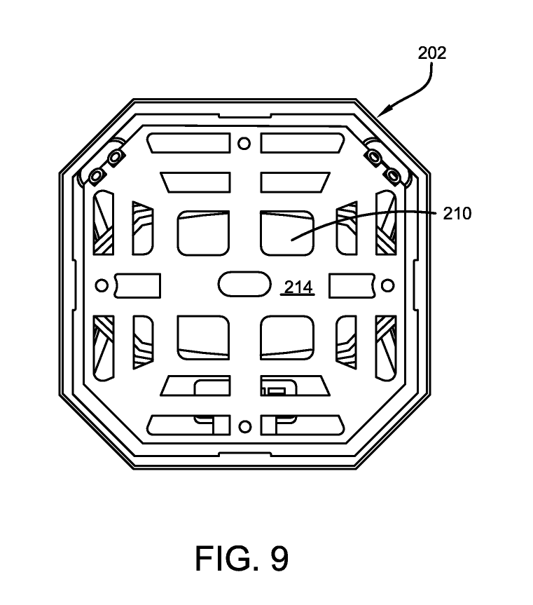

The present invention also provides an air freshener base unit that can serve to deliver to a fragrance upon actuation of a dispenser 11 received in the base unit. This is shown in FIGS. 7-9, wherein the base unit is designated by the numeral 200. Base unit 200 includes a top portion 202 that mates with a bottom portion 204 through a snap fit represented at the communication of a rib 206 (shown on the bottom portion 204) and a channel 208 (shown on the top portion 202). A dispenser 11 fits within the top portion 202 and rests on a bottle support 210. The bottle support is biased upwardly by a spring 212, though any suitable biasing means can be employed to carry out the function disclosed herein. Particularly, the spring 212 extends between the bottle support 210 and a vented platform 214, which is best appreciated in FIG. 9. The vented platform 214 is formed as part of the top portion 212. In the embodiment shown, the bottom portion 204 receives an air freshener 216. In other embodiments, one or more portions of the air freshener base unit 200 are made of a plastic infused with a fragrance.

It would be appreciated that the interior of the base unit 200 defines a collapsible volume 218 that decreases when the bottle support 210 advances downwardly toward the vented platform 214 when a user presses on the discharge spout 34 of the dispenser 11. The base unit 200 includes a vent 220 such that air is forced out of the vent 220 as the collapsible volume 218 decreases. In light of the presence of the air freshener 216 (or the forming of one or more portions of the air freshener base unit 200 with a plastic infused with a fragrance), actuating the dispenser 11 delivers a dose of fragrance from the base unit 200. Notably, in other embodiments, the air freshener 216 need not be employed, and instead an interior portion of the base unit is infused with a fragrance to impart a scent to the volume of air in the collapsible volume 218. In some embodiments, the vent 220 is adjustable to select the direction at which the dose of fragrance leaves the collapsible volume.

Though the base unit 200 is shown as being made of two mating portions and is shown with a vented platform 214, it will be appreciated that it is merely sufficient that the base unit define a collapsible volume that decreases upon actuation of the dispenser. The two-piece base unit is provided in particular embodiments in order to allow the user to have access to the air freshener 216 to replace the same when exhausted. The vented platform 214 provides an area of receipt for the spring to avoid the spring interfering with the air freshener; however it will be appreciated that various other structures can be practiced in accordance with the teaching herein.

Notably, though a basic dispenser 11 is shown in FIGS. 7-9, it will be appreciated that the dispensing systems such as systems 10 and 110 could instead be employed in the base unit 200.

It is thus evident that customizable fluid product dispensing system constructed as described herein substantially improves the art. Only particular embodiment(s) have been presented and described in detail, and the invention should not be limited by the drawings or the description provided. For an appreciation of the true scope and breadth of the invention, reference should be made only to the following claims.

1. A personalized fluid product dispensing system comprising
(a) a container including a volume of a fluid product; and
(b) a pump adapted to be secured to the container, the pump including at least one plastic component that carries a substance for imparting a property to a dispensed fluid product.

2. The system of claim 2, wherein said container includes a product reservoir and said at least one plastic component is a dip tube carrying a fragrance and extending downwardly from said pump into said product reservoir, such that said property imparted to said fluid product is a fragrance.

3. The system of claim 1, further comprising a cover assembly, wherein said container further comprises a neck and said cover assembly includes a base portion positioned over said neck and a top portion positioned over a discharge spout of said pump.

4. The system of claim 3, wherein said top portion is movable relative to said base portion to allow actuation of said pump.

5. The system of claim 1, wherein said cover assembly includes at least one plastic component having an infused fragrance, the cover assembly acting as an air freshener.

6. The system of claim 1, wherein said property imparted to said fluid product is a fragrance, and said cover assembly includes a vignette that corresponds to the fragrance.

7. The system of claim 1, wherein said container contains a volume of hand cleaner, hand sanitizer, or hand lotion.

8. The system of claim 1, wherein said property imparted by said plastic component of said pump is selected from one or more of a fragrance, a color, a moisturizer, a sunscreen and an insect repellant.

9. A method of merchandising a fluid product dispensing system comprising the steps of:
offering a container for sale, the container including a volume of fluid to be dispensed and a cap;
offering a plurality of pumps for sale, each of the pumps including a plastic component infused with a different fragrance adapted to impart the fragrance to the fluid product being dispensed; and
offering a plurality of cover assemblies for sale, the plurality of cover assemblies having various colors to allow for personalization of the dispensing system, each cover assembly adapted to be secured to the pump or container.

10. The method of claim 9, wherein the container includes a volume of hand sanitizer, hand cleaner, or hand lotion.

11. The method of claim 9, wherein said plurality of cover assemblies each include a plastic component with an infused fragrance for providing an air freshening function, each of said plurality of cover assemblies providing a different fragrance.

12. The method of claim 11, further comprising the step of offering a plurality of vignettes for sale, the vignettes including a plurality of images or graphics for personalization of the cover assembly, wherein the vignettes correspond to the fragrances of the pump.

13. The method of claim 9, wherein said plurality of pumps each include a dip stick extending downwardly from the pump and adapted to be in fluid communication with the fluid in the container, the dip stick having an infused fragrance to impart the fragrance to fluid passing therethrough.

14. The method of claim 9, further comprising the step of offering a plurality of vignettes for sale, the vignettes including a plurality of images or graphics for personalization of the cover assembly.

15. A dispensing system providing a dose of fragrance upon actuation of a dispenser thereof that is actuated to dispense product, the dispensing system comprising:
an fragrance-delivering assembly defining a collapsible volume that decreases upon actuation of the dispenser portion, said fragrance-delivering assembly carrying a fragrance; and
an outlet communicating with said collapsible volume such that the decreasing of the collapsible volume forces a does of air out at said outlet, said dose of air being scented by said fragrance.

16. The dispensing system of claim 15, wherein the dispenser includes:
a container having a neck, and
a pump having a discharge spout that is moved to actuate the dispenser, wherein said fragrance-delivering assembly includes a cover assembly including a base portion positioned over said neck and a top portion positioned over said discharge spout, said top portion and said base portion defining said collapsible volume, and said top portion being pressed toward said base portion to move said discharge spout and actuate said dispenser.

17. The dispensing system of claim 15, wherein the dispenser includes a container and the dispensing system further comprises:
a base unit defining said collapsible volume, said base unit including a bottle support mounted for reciprocal movement in said base unit, said container being supported on said bottle support such that actuation of the dispenser causes movement of said bottle support, said movement causing a decrease in said collapsible volume.

18. The dispensing system of claim 15, wherein the fragrance is provided by an air freshener carried in the fragrance-delivering assembly.

19. The dispensing system of claim 15, wherein the fragrance is carried by a plastic component of said fragrance-delivering assembly, said plastic component being infused with a fragrance.

20. The dispensing system of claim 15, wherein said outlet is an adjustable vent.
